(19) United States
(12) Patent Application Publication
(10) Pub. No.: US 2020/0297276 A1
(43) Pub. Date: Sep. 24, 2020

(54) Method for the Prevention of Brain Damage after Traumatic Brain Injury by Pharmacological Enhancement of KCNQ Potassium Ion Channels in Neurons

(71) Applicant: ADVANCED NEURORESEARCH THERAPEUTICS, L.L.C., Austin, TX (US)

(72) Inventors: Mark S. Shapiro, San Antonio, TX (US); Fabio A. Vigil, San Antonio, TX (US)

(21) Appl. No.: 16/799,787

(22) Filed: Feb. 24, 2020

Related U.S. Application Data
(63) Continuation of application No. 16/749,960, filed on Jan. 22, 2020.
(60) Provisional application No. 62/822,752, filed on Mar. 22, 2019.

(51) Int. Cl. A61B 5/00; A61K 31/222; A61B 5/04

(52) U.S. Cl. CPC A61B 5/4848; A61B 5/04001; A61K 31/222; A61B 5/4064

(57) ABSTRACT

A method for the prevention of brain damage and dysfunction after blunt or blast types of TBI by a single systemic dose application either intravenous (i.v.) or intraperitoneal (i.p.) of a pharmacological “opener” of KCNQ (“M-type”) potassium ion channels in brain.

### CROSS REFERENCE TO RELATED APPLICATIONS

This continuation application claims priority to and the benefit of U.S. non-provisional application Ser. No. 16/749,960, filed Jan. 22, 2020, and entitled “Method for the Prevention of Brain Damage after Traumatic Brain Injury by Pharmacological Enhancement of KCNQ Potassium Ion Channels in Neurons,” which claims priority to and the benefit of U.S. provisional application Ser. No. 62/822,752, filed Mar. 22, 2019, and entitled “Strategies of Targeting Electrical Signaling Proteins of Neurons to Prevent Acquired Epilepsies and Brain Dysfunction after Traumatic Brain Injury,” both of which are incorporated by reference herein.

### STATEMENT REGARDING FEDERALLY SPONSORED RESEARCH OR DEVELOPMENT

This invention was made with Government support under Grant No. W81XWH-15-1-0284 awarded by the U.S. Department of Defense. The Government has certain rights in this invention.

### BACKGROUND OF THE INVENTION

### Field of the Invention

The present invention relates to traumatic brain injury (TBI). More specifically, the present invention relates to a method for the prevention of brain damage and dysfunction after blunt or blast types of TBI by a single systemic dose application (i.v. or i.p.) of a pharmacological “opener” of KCNQ (“M-type”) potassium ion channels in brain.

### 2. Description of the Related Art

Nearly 3 million people in the U.S.A. suffer a traumatic brain injury (TBI) yearly. However, there are no pre- or post-TBI treatment options available to prevent brain damage after a TBI, leading to widespread neurological disease, such as seizures/epilepsy, psychiatric disorders, and suicides. The present invention is the first treatment to prevent these pathological outcomes, and may be administered hours after a TBI is sustained. KCNQ2-5 voltage-gated K⁺ channels underlie the neuronal “M current,” which plays a dominant role in the regulation of neuronal excitability. Prevention of TBI-induced brain damage is predicated on the hyper-excitability of neurons induced by TBIs and the decrease in neuronal excitation upon pharmacological augmentation of M/KCNQ K⁺ currents.

### BRIEF SUMMARY OF THE INVENTION

The present invention is a method for the prevention of brain damage and dysfunction after blunt or blast types of traumatic brain injury (TBI) by a single systemic dose application (i.v. or i.p.) of a pharmacological “opener” of KCNQ (“M-type”) potassium ion channels in brain. Such brain damage and dysfunction can include post-traumatic seizures, a mal-adaptive inflammatory response, microgliosis, astrogliosis, widespread neuronal death, breakdown of the blood-brain-barrier, post-traumatic epilepsy, cognitive and locomotor dysfunction, mood changes, early Alzheimer's Disease and loss of quality of life. The present invention was included in an article entitled “Prevention of brain damage after traumatic brain injury by pharmacological enhancement of KCNQ (Kv7, “M-type”) K⁺ currents in neurons,” Journal of Cerebral Blood Flow & Metabolism, 2019, incorporated by reference herein.

The present invention does not concern one particular opener molecule, but rather to the approach that is most likely applicable to many of them. Such opener molecules include retigabine (RTG), (2-amino-4-(4-fluorobenzylamino)-1-ethoxycarbonylaminobenzene, an aminoalkyl thiazole derivative (known as Ezogabine in Europe and sold under the trade name, Potiga, formerly FDA approved but now off the market as an anti-convulsant), ICA-069673 ([N-(2-chloro-5-pyrimidinyl)-3,4-difluorobenzamide]) and ICA-27243, and its derivatives, (2-amino-4-(arylamino-phenyl) carbamates (SF0034, RL648_81), and its derivatives, and other such “openers” (agonists) of KCNQ2-5 potassium ion channels that increase their currents in brain neurons.

Seizures are very common after a TBI, making further seizures and development of epilepsy disease more likely. The present invention demonstrates that TBI-induced hyperexcitability and ischemia/hypoxia lead to cellular metabolic stress, cell death and a maladaptive inflammatory response that causes further downstream morbidity, and development of long-term epilepsy. Using a mouse controlled closed-cortical impact blunt TBI model, and the “blast-tube” TBI model, systemic administration of the prototype M-channel “opener,” retigabine (RTG), 30 min after TBI, reduces the post-TBI cascade of events including spontaneous seizures, enhanced susceptibility to further seizures, cellular metabolic stress, harmful immunological/inflammatory responses, blood-brain barrier (BBB) breakdown, neuronal death, neuronal necrosis/apoptosis and development of long-term epilepsy disease.

There are currently no treatments to prevent brain damage after a TBI, leading to widespread neurological disease, such as seizures/epilepsy, psychiatric disorders, and suicides. The present invention is the first treatment that will prevent these pathological outcomes, and may be applied hours after a TBI is sustained.

Currently, there are no medical treatments for either kind (blunt or blast) of TBI whatsoever, and thus, widespread mortality and morbidity numbering in the millions is the result. Thus, the present invention is the first treatment after a TBI event to prevent brain damage and dysfunction for millions of Americans and tens of millions of individuals worldwide. Thus, the present invention represents a completely new and novel approach for treatment of TBI that has been tested by no other lab or entity, worldwide.

### BRIEF DESCRIPTION OF THE SEVERAL VIEWS OF THE DRAWINGS

FIG. 1 is a schematic of the chain of events of an embodiment of the present invention.

FIG. 2 depicts general characteristics of M channels as used in the present invention.

FIG. 3 shows graphs tracking action potential firing in response to various stimuli as used in the present invention.

FIGS. 4A through 4E depict graphs and electroencephalography (EEG) recordings documenting experimental support of the present invention.

FIGS. 5A through 5D show action potential recordings and resulting data supporting the present invention.

FIG. 6A through K depict graphs documenting experimental support of the present invention.

FIGS. 7A and 7B depict graphs documenting experimental support of the present invention.

FIG. 8 depict a graph demonstrating dentate gyrus responses to kainic acid between sham, TBI and TBI+RL-648_81 treated mouse groups of the present invention.

FIG. 9 depicts in vivo imaging of intracellular calcium levels in living brain nerve cells in vivo, as a reporter of activity/hyperactivity.

FIG. 10 shows graphs and in vivo images recording the optical measurement in living mice in vivo of extracellular glutamate [glutamate]ₒ by viral expression of a fluorescent sensor in support of the present invention.

FIG. 11 shows a graphical representation of the long-term effects of TBI, wholly distinct from treating seizures with anti-convulsants, showing the probability of seizures for different groups of mice tested using the present invention.

### DETAILED DESCRIPTION OF THE INVENTION

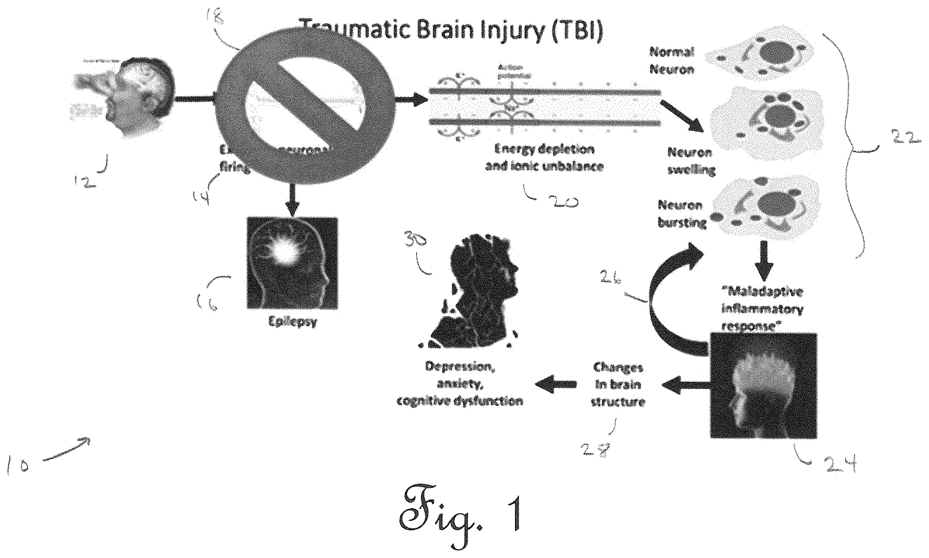

FIG. 1 depicts the chain of events 10 of the present invention. An example of a traumatic brain injury may be as a result from the impact of a first or blunt instrument to the front of someone's head 12. This TBI may result in a concussion and also post concussive syndrome with significant, persistent headaches and blurred vision, and short term memory loss. Excessive neuronal firing 14 commences. If continued, then post-traumatic seizures and epilepsy 16 may result. If, however, excessive neuronal firing 14 does not cease, next follows the depletion of intracellular energy stores and ionic imbalance event 20. This may continue until healthy neurons begin to swell and ultimately burst, as shown in event 22 in FIG. 1. A maladaptive inflammatory response 24 occurs. From here, either further swelling and bursting of neurons may continue, as shown by return loop 26. Changes in the brain structure and proper functioning 28 become evident, leading to the final event 30 of depression, anxiety and cognitive dysfunction.

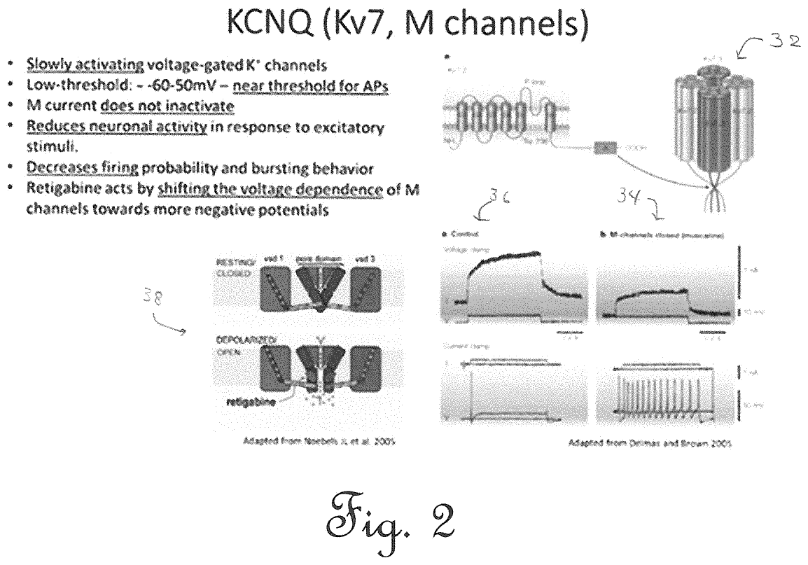

Referring now to FIG. 2 the general characteristics of M channels 32, which are tetramers of four subunits, most often KCNQ2 & KCNQ3, are as shown. Current waveforms 34 and 36 are shown before and during stimulation of muscarinic acetylcholine receptors (top) 34 and the greatly induced action potential (AP) firing 36 when M current is reduced as shown.

Slowly activating voltage-gated K⁺ channels have a low threshold of ˜−60 to −50 mV—near the threshold for action potentials. The M current 32 does not inactivate, but instead reduces neuronal activity in response to excitatory stimuli which decreases firing probability and bursting behavior. Retigabine and similar M-channel “openers,” act by shifting the voltage dependence of M channels towards more negative potentials, as shown in representation 38 of FIG. 2.

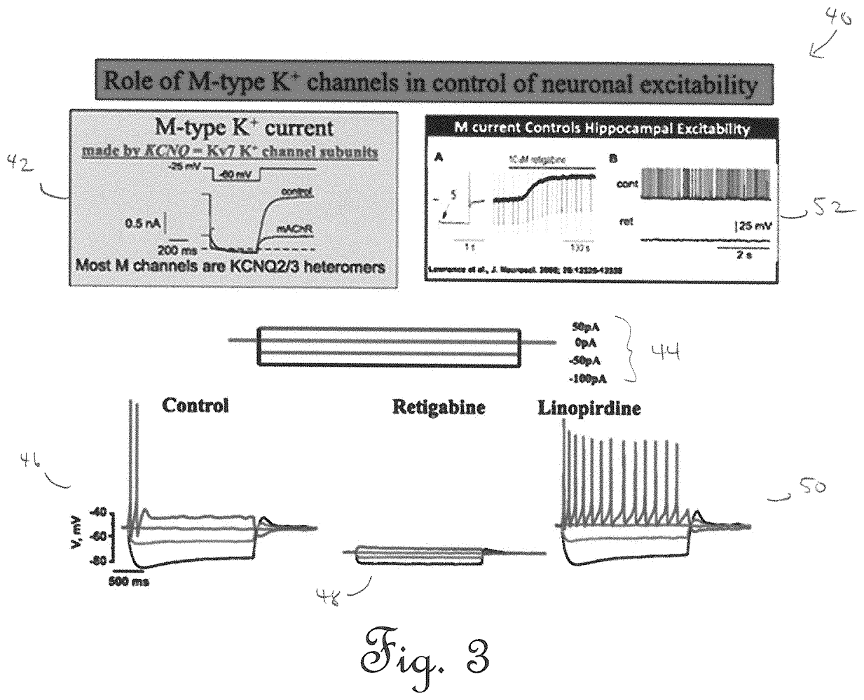

Turning now to FIG. 3, the role 40 of M-type K⁺ channels in control of neuronal excitability is discussed. M-type K⁺ current is provided by KCNQ Kv7 K⁺ channel subunits, as depicted by representation 42. Various currents are applied 44 ranging from 50 pA to −100 pA, and the resulting action potential firing recorded.

Graph 48 shows action potential (AP) fires in response to stimuli in a sympathetic neuron when M current is enhanced by the “opener” drug retigabine. Complete silencing of the neuron occurs, as shown by graph 48. When M channels are completely blocked by the M-channel “blocker,” linopirdine, the result is out-of-control AP firing as when a seizure occurs, or when retigabine is applied to a “brain slice” of the hippocampus in brain, as shown by graphs 52, again converting a rapidly-firing neuron into one totally quiescent. Thus, manipulation of M-channel activity profoundly affects excitability, and a pharmacological “opener” can act as a potent brake on hyperexcitability.

The present invention suggests that acutely reducing neuronal excitability and cellular energy demand via M-current enhancement is a novel model of therapeutic intervention against post-TBI brain damage and dysfunction. This is done by reducing the initial TBI-induced hyperexcitability before seizures occur that only exacerbate cellular energy depletion, thus, “nipping in the bud” the deleterious chain of events that cause a TBI to be so damaging to the brain (see, e.g., FIG. 1).

As discussed further below, the newer M-channel opener, RL-648_81, applied 30 min after a blunt TBI, reduces the increased seizure susceptibility in brain slices, ex vivo. RL-648_81 also prevents TBI-induced hyper-excitability in cortex when neuronal activity is imaged in vivo through a cranial window in a living mouse brain. Finally, experimentation demonstrates that brain damage manifesting in long-term epilepsy occurs after 3-consecutive mild-moderate blast TBIs, and that such is prevented by M-channel augmentation, a series of events often experienced by members of the armed services. Administration of an M-channel opener once after each mild TBI will abolish the deleterious effects on the brain.

In experiments, the present invention modeled mild blast TBI injury in mice to observe effects on neuronal excitability. Mice were exposed to an average of 14.6±0.5 psi (100 kPA±3.4 kPa) maximum peak overpressure, which has been classified as mild blast TBI (0-145 kPa) in rats and mice using a number of physiological parameters. Experiments were first performed to evaluate the effect of the blast TBI model, with a single exposure, or with repeated second and third exposures using 24-hour intervals between TBI.

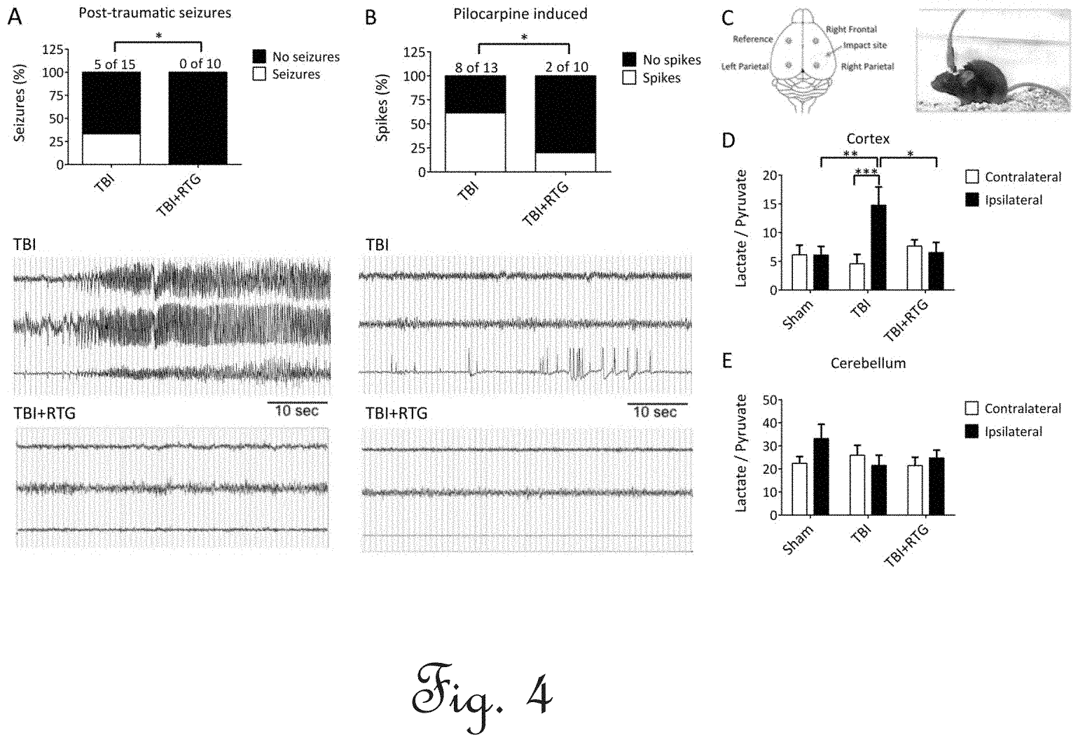

Referring now to FIGS. 4A through 4E, as an indicator of blunt TBI-induced seizures and TBI-induced seizure susceptibility, mice were continually monitored by video and EEG. The present invention shows that, whereas mice subjected to TBI and no drug, 33% of mice displayed post-traumatic seizures, of those mice subjected to TBI and the M-channel opener, retigabine (RTG), zero mice displayed a post-traumatic seizures, as shown in FIG. 4A. In addition, whereas mice subjected to TBI and no drug, as an indicator of cellular metabolic stress, the lactate/pyruvate ratios in cortex and cerebellum were increased by nearly 3-fold in the same side of brain as the trauma, whereas those subjected to TBI and RTG 30 min later, there was no significant increase in cellular metabolic stress, as shown in FIGS. 4D and E, respectively.

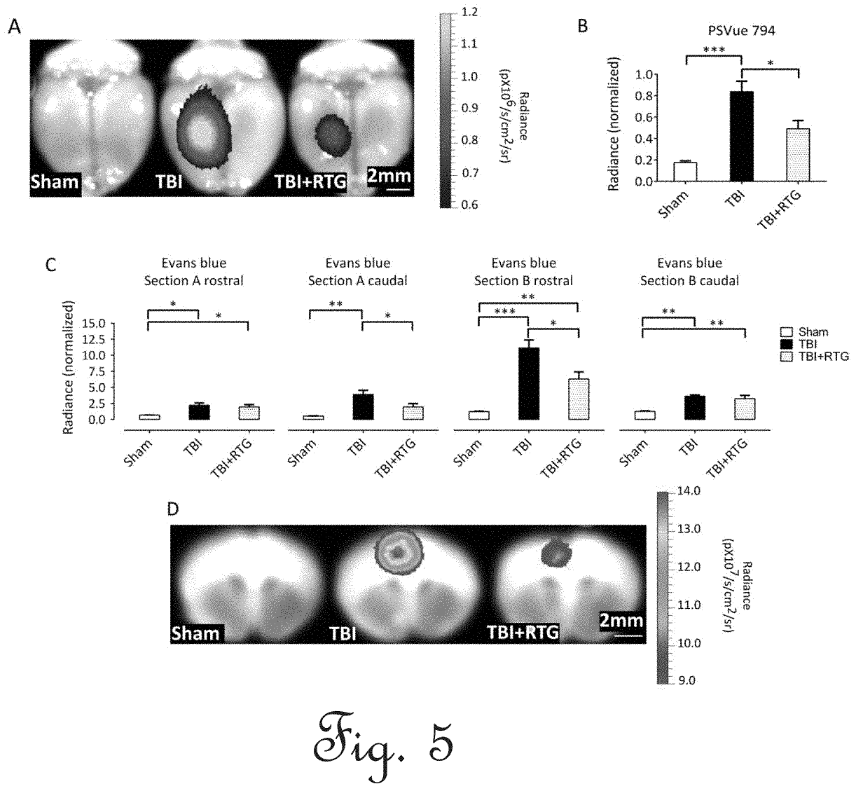

Referring now to FIGS. 5A-D, as an indicator of breakdown of the Blood-Brain Barrier (BBB), the Evans blue extravasation assay at 72 h post-TBI showed a dramatic breakdown of the BBB in mice not treated with any drug, but in mice subjected to TBI and then treated with RTG 30 min later, the breakdown of the BBB was reduced by about 50%, as shown in FIG. 5C.

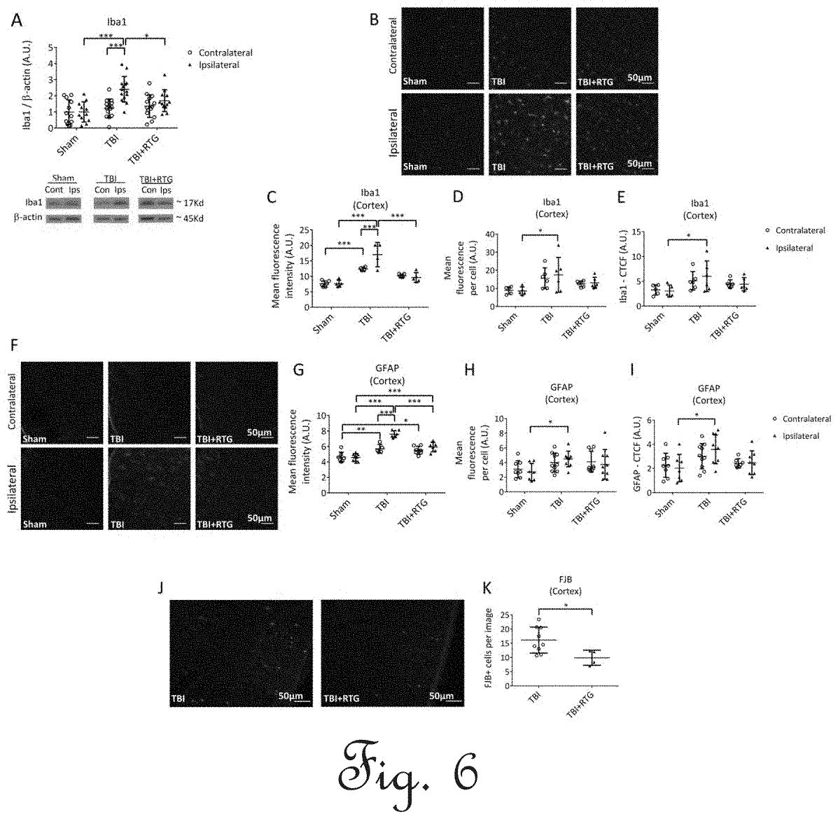

The following four sections concern the blockade of post-TBI mal-adaptive immunological/inflammatory responses block by augmentation of M current following TBI. Referring now to FIGS. 6A-C, as an indicator of microgliosis, the biomarker ionized Ca²⁺ binding adaptor molecule 1 (Iba1) was assayed, both by western blot and by immunofluorescence. In mice subjected to TBI but no drug, Iba1 expression was increased by about double on the ipsilateral side of the brain to the trauma, but in mice subjected to TBI and RTG 30 min later, there was no such increase in either type of Iba1 assay.

FIGS. 6A through 6K demonstrate M-current augmentation suppressed TBI-induced microglial activation in the ipsilateral cortex. With specific reference now to FIGS. 6F-I, as an indicator of TBI-induced astrogliosis, the levels of glial fibrillary acidic protein (GFAP) were assayed after TBI by immunofluorescence. In mice subjected to TBI but not drug, GFAP levels were increased by nearly double in cortex on both sides of the brain, but in mice subjected to TBI followed by RTG 30 min later, there was no such increase.

Referring now to FIG. 6J, the fluorescent dye, Fluoro Jade B (FJB) labeling was used as an indicator of TBI-induced death of neurons. In mice subjected to TBI but not drug, there was substantial labeling of neurons throughout the brain, indicating substantial TBI-induced death of neurons. But in mice subjected to TBI and RTG 30 min later, labeling of brain neurons by FJB was minimal, as shown in FIG. 6K.

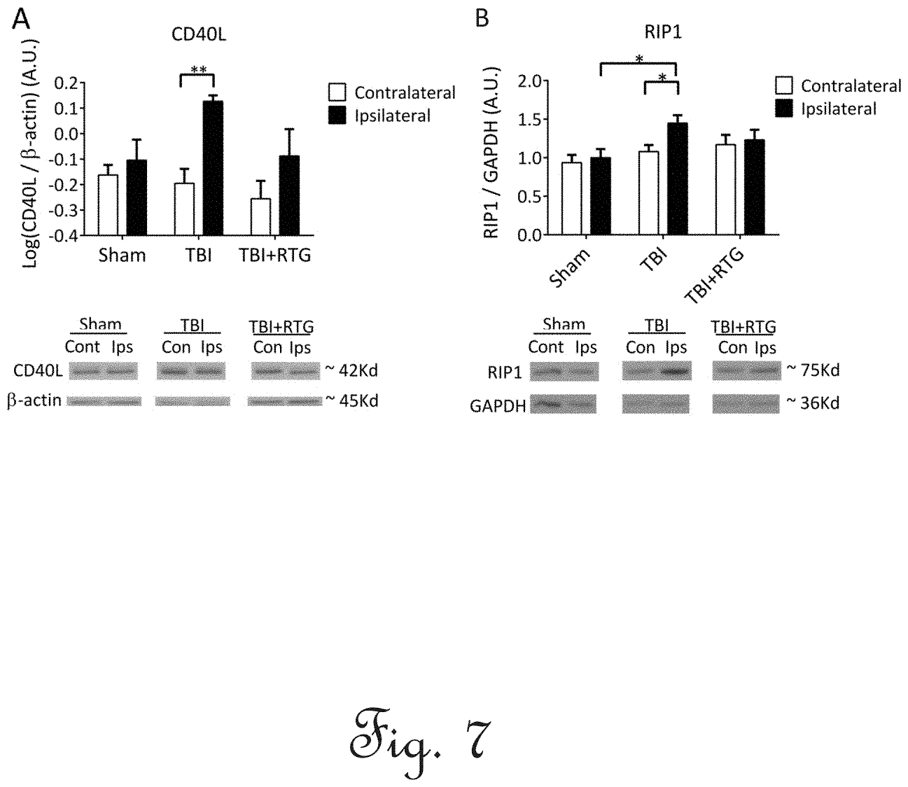

Referring now to FIG. 7A, as an assay of the mal-adaptive immune response using the biomarker inflammation-linked CD40 ligand (CD40L), mice subjected to a blunt TBI but no drug exhibited over a two-fold increase in levels of CD40L over that of sham mice or mice subjected to TBI but not drug However, in mice subjected to a blunt TBI followed after 30 min by RTG, the levels of CD40L were not significantly different than in sham mice.

Referring now to FIG. 7B, as an assay of necrosis and apoptosis of neurons, biomarker receptor-interacting protein 1 (RIP-1) levels in brain were measured. In mice subjected to TBI but not drug, there was a 50% increase of RIP-1 levels in brain, indicating substantial TBI-induced necrosis and apoptosis. But in mice subjected to TBI and RTG 30 min later, the levels of RIP-1 in brain were not significantly different than shams.

In another embodiment of the present invention, ex vivo imaging of intracellular calcium levels in neurons, which are a reporter of activity or hyper-activity of neurons, were assayed in the brain slices using transgenic mice expressing the genetically encoded indicator of calcium levels, GCaMP6f only in neurons. As seen in FIG. 7, such transgenic mice were used to image intracellular calcium levels inside critical subsets of brain neurons using ex vivo brain-slice imaging experiments.

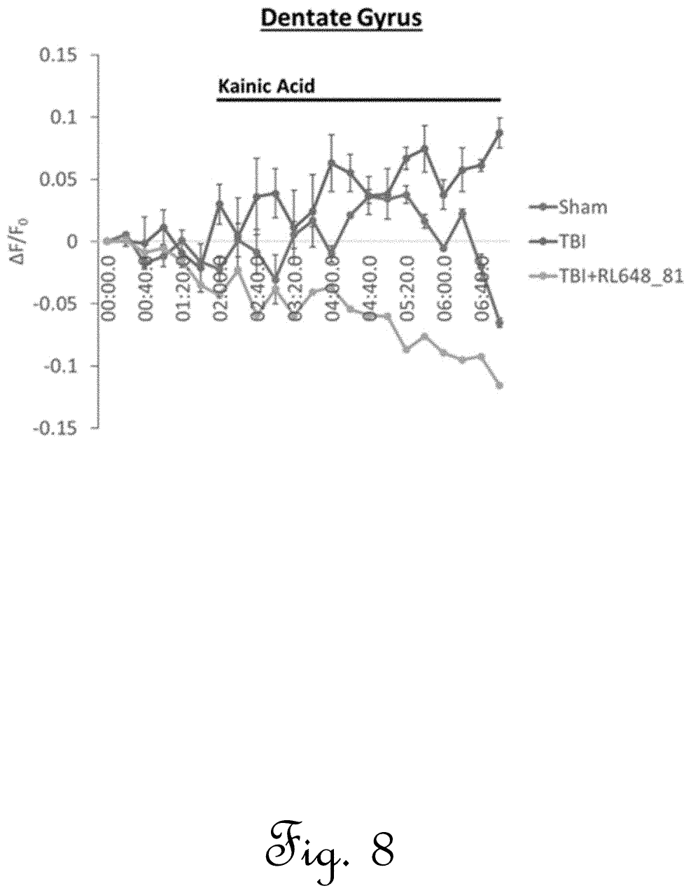

Turning now to FIG. 8, the chemoconvulsant, kainic acid (KA), was used to stimulate neurons from brain slices of the dentate gyrus (DG) of the hippocampus (the part of the brain where most new memories are thought to form) from sham mice, mice subjected to a TBI but not drug or TBI followed by RL-648_81. RL-648_81 is a new generation drug that facilitates M-current solely produced by heteromeric tetramers of KCNQ2&3 subunits, the subunits found predominantly in brain neurons, thus sparing side-effects of RTG on smooth muscle, such as that in bladder, which causes urinary incontinence.

Using this technique, it was discovered that M-current augmentation by RL-648_81 treatment occludes TBI-induced increased hyperexcitability in response to KA stimulation. The experiments show the hippocampus to have a stronger TBI-induced increase in hyper-activity in response to KA stimulation, which was completely abolished by RL-648_81 treatment 30 min after the TBI. Thus, the present invention concerns not only one molecule, but rather the general approach of preventing TBI-induced brain damage, which has never before been postulated or demonstrated.

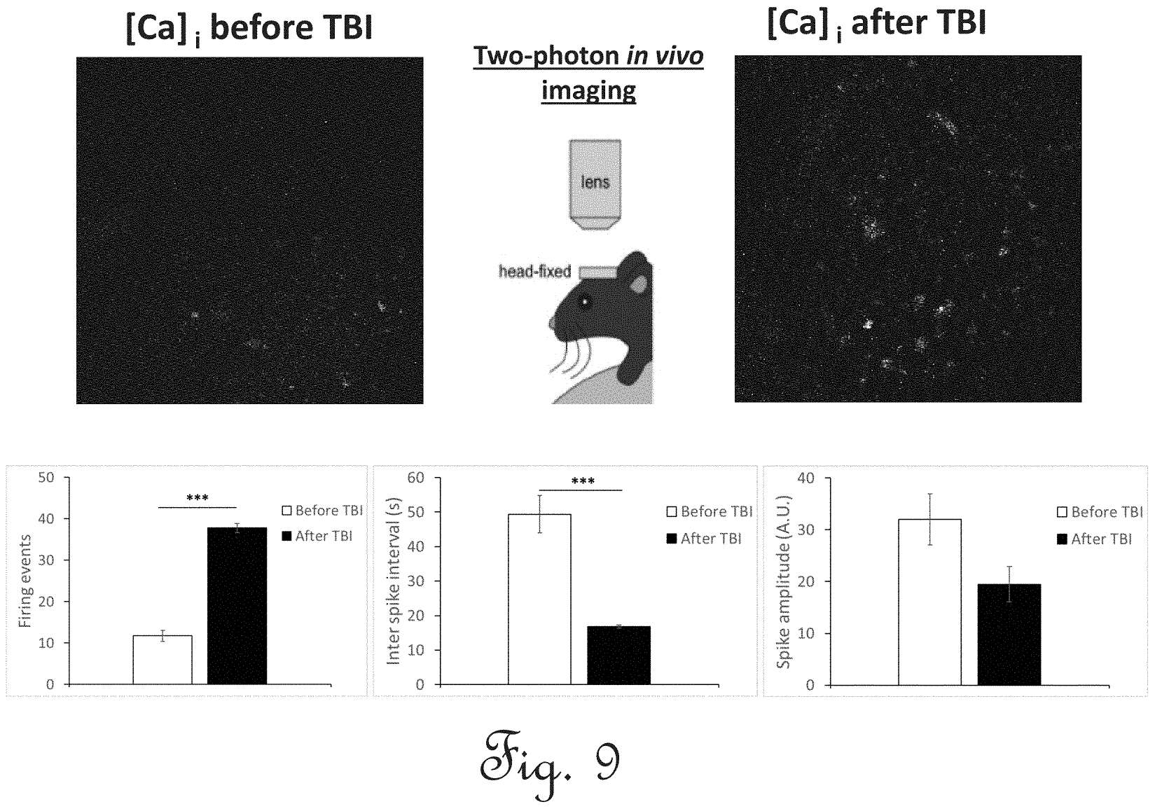

Referring now to FIG. 9, another embodiment of the present invention concerns in vivo imaging of intracellular calcium levels in neurons from living brain in vivo, as a reporter of activity/hyperactivity. The genetically-encoded calcium reporter, GCaMP6f, was used in transgenic mice in which the reporter is expressed in critical subsets of brain neurons, allowing in vivo recordings of neurons of living brain, as a reporter of activity/hyperactivity in cortex induced by TBI, the first event in the hypothesis of the present invention, which causes TBI-induced brain damage (See, e.g., FIG. 1). For these experiments two-photon confocal live imaging of mouse cortical neurons through a cortical window was used. Live video records of the activity of cortical neurons in mice before and after TBI were made. As can be seen in the graphs presented in FIG. 9, a significant increase in neuronal firing and a decrease in inter spike interval was observed after a blunt TBI.

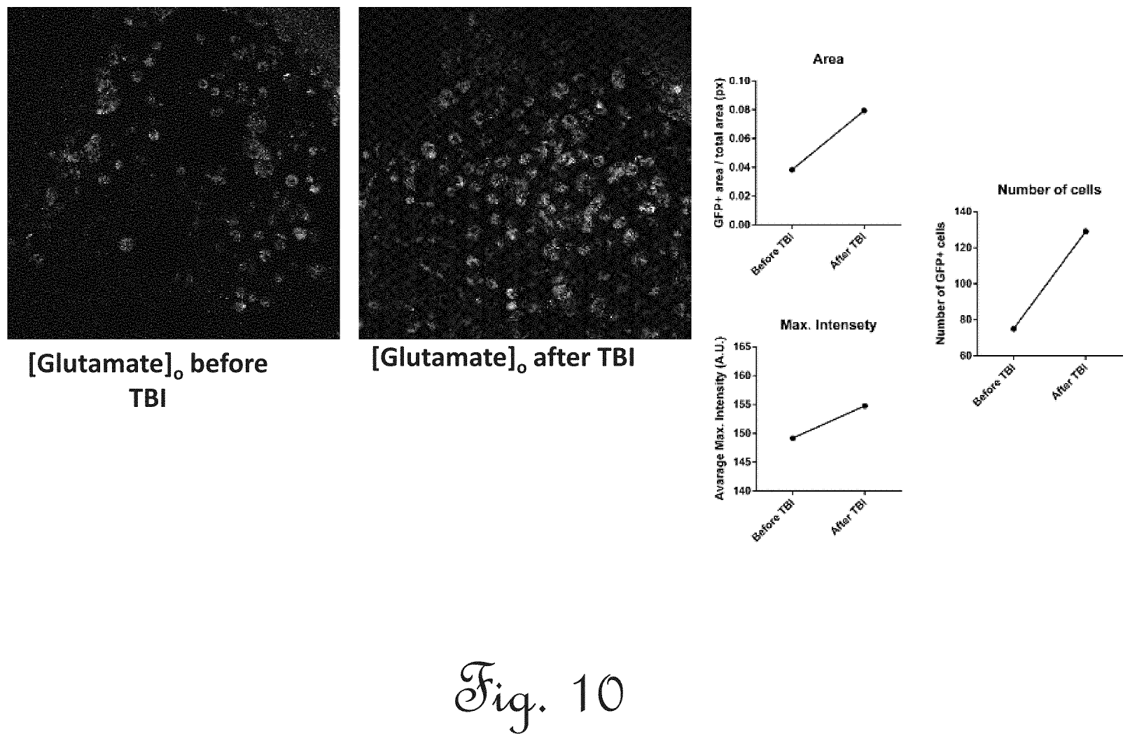

Another embodiment of the present invention, and referring now to FIG. 10, concerns the optical measurement in living mice in vivo of extracellular glutamate [glutamate]ₒ, the major excitatory neurotransmitter in the brain, by viral expression of a fluorescent sensor, iGluSnFR packaged in the viral vector AAV-PHP.B-Syn1, injected i.v. into the mice. Again, this sensor may only be expressed in brain neurons, as was the case for GCaMP6s/f. It can be seen that in mice subjected to a TBI, [glutamate]ₒ is strongly increased to abnormal levels. These results support the hypothesis of the present invention regarding TBI-induced hyper-excitability as the first event in the cascade of events resulting in brain damage and dysfunction.

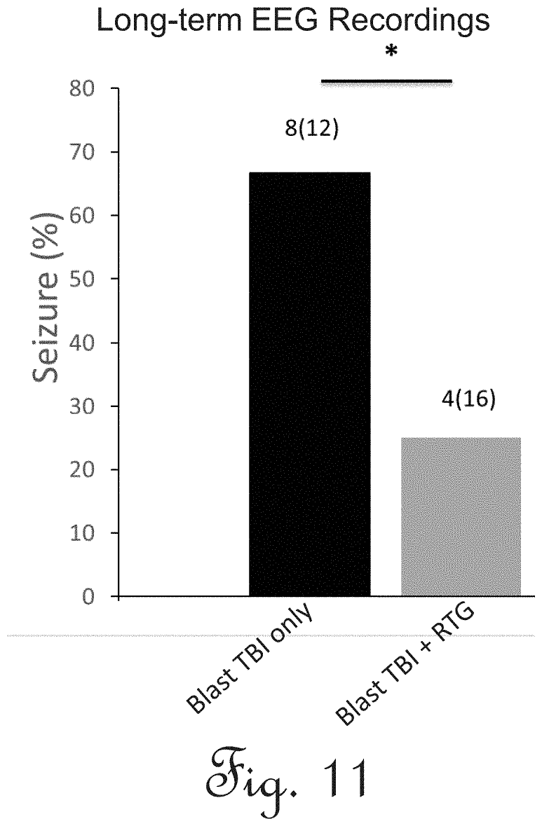

In another embodiment of the present invention, and now referring to FIG. 11, the long-term effects of TBI are shown, in particular the development of epilepsy disease, which is wholly distinct from treating seizures with anti-convulsants. The present invention also addresses the case of repetitive, moderate, blast TBIs, such as those experienced routinely by members of the armed forces. Mice were subjected to three blast TBIs, one day apart, either with no drug application, or with application of RTG 30 min after each one. As can be seen, 67% of mice displayed spontaneous seizures over a long period of time, as observed by electroencephalogram (EEG) and video recording from the period of 1 month to 12 months after injury. The occurrence of these seizures confirms the occurrence of post-traumatic epilepsy (PTE) after repetitive blast-TBI. RTG treatment 30 min after each blast TBI was able to significantly reduce PTE occurrence many months to a year later. Only 25% of the RTG treated mice developed PTE. Eight sham animals were also monitored and no seizures were observed. There is currently no FDA-approved treatment for PTE. Hence, treatment with M-channel openers represents a paradigm change in PTE prevention.

Brain damage, deficits and dysfunction resulting from commonly occurring TBIs can be prevented by acute administration of a KCNQ/Kv7/M-type potassium channel opener shortly after the occurrence of a TBI. The TBI can be of the blunt type or the blast/shockwave type, as commonly experienced in falls, impact of the head with a hard object, vehicular accidents, explosions on the battlefield in the vicinity of military service personnel, or shock waves from the use of large weapons near the victim by either a fellow service member, or the enemy. Even very mild TBIs, although not perceptible to the victim, when repeated multiple times, cause brain damage, which can also be prevented by M-channel pharmacological openers when administered after each TBI, or after a succession of TBIs.

The present invention has application in the treatment of brain injuries caused as a result of engaging in contact sports/athletic events, in which head impacts are common, including American football, soccer, ice hockey, boxing, bicycle falls and the like.

The various embodiments described herein may be used singularly or in conjunction with other similar methodologies. The present disclosure includes preferred or illustrative embodiments in which a method for the prevention of brain damage and dysfunction after blunt or blast types of TBI by a single systemic dose application (i.v. or i.p.) of a pharmacological “opener” of KCNQ (“M-type”) potassium ion channels in brain is described. The present invention is intended to cover the entire spectrum of M-channel “openers,” including those listed herein, and any new compounds or derivatives of existing compounds that may be developed on this same target. Alternative embodiments of such a method can be used in carrying out the invention as claimed and such alternative embodiments are limited only by the claims themselves. Other aspects and advantages of the present invention may be obtained from a study of this disclosure and the drawings, along with the appended claims.

I claim:

1. The method of preventing brain damage and dysfunction after traumatic brain injury, said method comprising the steps of:
administering a dose of a pharmacological opener to an organism;
monitoring said organism for a predetermined time;
observing neuronal activity; and
recording said neuronal activity.

2. The method of claim 1 wherein in said administering step, said pharmacological opener is a KCNQ/Kv7/M-type potassium channel opener.

3. The method of claim 2 wherein said administering step is performed shortly after the occurrence of said traumatic brain injury.

4. The method of claim 3 wherein the neuronal traumatic brain injury-induced hyper-excitability and cellular energy demand are reduced via M-current enhancement.

5. The method of claim 4 wherein said dose in said administering step is limited to a single dose after said traumatic brain injury.

6. The method of claim 5 wherein in said administering step, said pharmacological opener is retigabine and its derivatives.

7. The method of claim 5 wherein in said administering step, said pharmacological opener is RL-648_81 and its derivatives.

8. The method of claim 6 wherein said traumatic brain injury is of blunt traumatic brain injury.

9. The method of claim 7 wherein said traumatic brain injury is of blunt traumatic brain injury.

10. The method of claim 6 wherein said traumatic brain injury is of blast traumatic brain injury.

11. The method of claim 7 wherein said traumatic brain injury is of blast traumatic brain injury.
